(19) United States
(12) Patent Application Publication
(10) Pub. No.: US 2015/0233884 A1
(43) Pub. Date: Aug. 20, 2015

(54) Sampling and Analytical Platform for the Remote Deployment of Sensors

(71) Applicant: Scott Russell Burge, Tempe, AZ (US)

(72) Inventor: Scott Russell Burge, Tempe, AZ (US)

(21) Appl. No.: 14/183,014

(22) Filed: Feb. 18, 2014

(51) Int. Cl. G01N 33/18; G01J 3/50; G01N 27/333; G01N 35/10; G01N 27/30

(52) U.S. Cl. CPC G01N 33/1826; G01N 35/1065; G01N 33/182; G01N 33/18; G01N 27/302; G01N 27/333; G01N 27/30; G01J 3/50; G01N 2035/00306

(57) ABSTRACT

An automated microprocessor-controlled monitoring system for the sampling and analysis of environmental contamination has independent multiple sample chambers 47, 48. The sample chambers are populated with multiple analytical sensors 59, 60, 61, Multiple water level sensors 49, 50, 135 located in the sample chambers are capable of determining the volume of sample, or standard, introduced into the individual sample chambers. The monitoring system is standardized with independent calibration modules 7L 80, 89 to support the analytical sensors in the sample chambers. This configuration of a monitoring system allows a “plug and play” configuration with all analytical sensors capable of standardization. The system anticipates the incorporation of future sensing methodologies through its flexible design. The disclosed system is capable of operating multiple pumps 17, 18, 19 and measuring the water levels 30, 31, 32 in multiple monitoring wells 36, 37, 38 allows for the automated acquisition of data for slug, aquifer, and tracer tests. Other embodiments are described and illustrated.

### CROSS-REFERENCE TO RELATED APPLICATIONS

Reference is made to our Provisional Application No. 61/766745 filed Feb. 20, 2013 entitled “Sampling and Analytical Platform for Remote Deployment of Sensors” by the present inventors.

### BACKGROUND OF THE INVENTION

1. Field of Invention

The field of environmental monitoring has many chemical parameters and environmental contaminants to be measured for the purposes of environmental compliance. The environmental contaminants to be monitored will vary based on the industry or site to be assessed. The cost of developing monitoring systems tar each type of industry or contaminated site is prohibitive, therefore, an automated monitoring system that can be configured using a series of independent chambers with analytical sensors and calibration modules combined into a package that will measure many of the important parameters of a facility would have the ability to attract a significant share of the environmental monitoring market.

This invention relates generally to the art of the automated sampling and analysis of environmental contaminants in water or atmospheres at unattended locations. Unattended locations include municipal water treatment facilities and/or groundwater investigations. The invention describes a monitoring system with multiple independent chambers with the ability of measuring the volume of the sample introduced into each of the chambers. Analytical sensors are exposed to the samples contained in the interior of each of the chambers. The monitoring system is capable of calibrating each of the sensors located in the multiple chambers with independent calibration modules. The system allows for the design of a “plug and play” monitoring system. This flexibility allows a user to design a customized monitoring system for the environmental contaminants of interest at their facility.

2. Background-Prior Art

The following is a tabulation of some prior an that is relevant

### U.S. Patents

| Patent No. | Kind Code | Issue date | Patentee |
|---|---|---|---|
| 5,646,863 | A | July 1997 | Morton |
| 6,021,664 | | Feb. 8, 2000 | Granato et al. |
| 6,936,156 | B2 | Aug. 30, 2005 | Smith et a1. |
| 7,247,278 | B2 | Jul. 24, 2007 | Burge et al. |

3. Discussion of Prior Art

The field of automated monitoring systems is mature with a history of prior art spanning over 30 years and many commercialized versions readily available on the current market. The advent of automated instrumentation was made possible by the availability of affordable microprocessors in the early 1980s. The prior art describing monitoring methods supporting multiple sensors include:

U.S. Pat. No. 7,247,278 describes a monitoring system to transfer groundwater samples from a well to an analytical sensor located at the surface, and methods for calibrating the sensor at the surface. There is no disclosure of multiple analytical chambers, multiple calibration systems or deployment of alternative sensors.

U.S. Pat. No. 5,646,863 describes a monitoring system that has a series of flow-through measuring cells for measuring multiple analytes, however, the invention does not describe interchangeable chambers, interchangeable calibration modules, or the measurement of the sample volumes delivered to the chambers. The sampling system flows the sample through the sample chamber for analysis by the analytical sensors. The description limits the capability of the number of analytical methods that may be performed by the system. The system does not allow for the expansion of the system for additional future sensors.

U.S. Pat. No. 6,021,664 describes a flow-through system that has one sample cell with several sensors (temperature, conductance, dissolved oxygen, pH and ammonia). No reference is made for measuring the volume in the sample cell, or to an interchangeable cell for other contaminants. Most of the disclosure is associated with purging groundwater wells. The sampling system flows the sample through the sample chamber for analysis by the analytical sensors.

U.S. Pat. No. 6,936,156 describes a flow-through system with the capability of recirculating through the sample cells. The system does not describe multiple sample chambers each capable of measuring the volumes delivered to the chamber. Additionally, the invention does not describe a method for incorporating additional sample chambers or cells, or the calibration of the sensors in the additional cells. The sampling system flows the sample through the sample chamber, or re-circulates the sample for analysis through the sample chamber.

Most of the commercial instruments (Hach) describe flow-through cells with the ability to calibrate the system by the injection of standards into the flow-through systems.

### BRIEF DESCRIPTION OF INVENTION

The invention described in this disclosure is a monitoring system composed of separate sample chambers with associated analytical sensors capable of determining the concentrations of important environmental contaminants in the environment. The system described has the ability to measure the volume of the sample, standards or reagents injected into the sample chambers. The environmental contaminants include biological, dissolved metals, anions, volatile and semi-volatile organics and radiologicals. Many of the prior art citations and current commercialized instruments use rigid flow-through systems that are not readily suited for many environmental analyses.

The deployment of analytical sensors (pH, ORP, conductivity, colorimetric, radiometric, etc.) at remote locations requires the sensors to be housed in an environmentally controlled space, provide power, control, and communication capabilities to operate the sensors, and transmit the data to remote users. A method of sampling must be provided to expose the sensors to the media being monitored such as natural waters, process water, or atmospheres. An important aspect of any analytical protocol is the ability to interrogate the sensors at frequent intervals using standards. The interrogation may be accomplished by using multiple calibration standards to create a calibration curve, or by using one standard to calculate a calibration factor.

This invention consists of a central sampling/analytical platform with the ability to connect additional analytical boards, various analytical sensors, alternative sample chambers and the accompanying calibration components as “plug and play” modules. This design recognizes that most analytical protocols, regardless of the sensor being employed, share similar tasks such as sample introduction, temperature control, communications, control, and cleaning. Therefore, the design of the sampling/analytical platform unifies all the operational components that are constant regardless of the type of analytical sensor being deployed. These common features include sampling, components, cleaning, communication, environmental controls, and power control. The basic design of the analytical platform does not include any specific sample chamber, analytical sensor, or method of sensor interrogation (introduction of standards). All three of these sensor-specific features are plug-in modules to the basic sampling/analytical platform. This relative freedom of the sampling/analytical platform from any specific analytical sensor allows the system to quickly be configured for many types of analytical sensors. This type of analytical platform design is independent of any specific type of sensor allowing the analytical platform to accommodate new sensor technologies as they become available.

In this disclosure, the analytical sensor and its accompanying analytical components (sample chamber and interrogation module) are all plug-in modules that are connected to the sampling/analytical platform when the specific analytical sensor is required. This concept is illustrated on FIG. 2. This figure illustrates that each analytical sensor has an accompanying interrogation (calibration) module and sample chamber. The interrogation component consists of valves and/or pumps that inject the standard solutions into the sample chamber for the purpose of standardization, or to check on the validity of a calibration factor. The analytical platform is designed to recognize the sensor and the accompanying interrogation module.

An example would be a pH electrode, accompanying sample chamber, and interrogation module. The electrical leads of the pH electrode are fabricated into a plug, or other type of connector, that connects to the electronics of the sampling/analytical platform, and the pH electrode is inserted into a sample chamber compatible with the analytical sensor and the platform. The interrogation module has its electronic components fabricated into a second plug, or other type of connector, that connects to the electronics of the sampling/analytical platform. The tubing for the delivery of the standard(s) is inserted into the same sample chamber housing the pH electrode. The operation of the sampling, calibration, and analysis using the pH probe is controlled by the microprocessor incorporated in the main board.

Once the three components (analytical sensor, sample chamber and interrogation module) are connected, the operation of the sampling/analytical platform allows for a complete analysis of water samples using the pH sensor including sampling, interrogation, quality control checks and cleaning.

It is possible that several sensors may use the same sample chamber. An example would be a single sample chamber accommodating a pH, ORP and conductivity sensors.

Sample chambers are designed to allow for the volumetric measurement of the different solutions introduced into the sample chamber for the purpose of diluting standards and/or reagents to aid in the analysis of the target analyte. The volume may be measured within a sample chamber using optical sensors, conductivity sensors or other methods to determine the volume of water in the sample chamber. The sample chambers may be fitted with stirring motors, or other methods for agitating the solution. Additionally the sample chamber may be fitted with methods of heating the chamber to establish a constant temperature during the analysis.

The addition of alternative analytical sensor modules with accompanying interrogation modules allows monitoring of additional parameters such as conductivity, ORP, etc. The design of this sampling/analytical platform is a flexible design that allows analytical sensors to be added (or incorporated) as they are developed without the costly requirement to redesign the platform to house the new analytical sensors.

The sample chamber is an important component of the sampling/analytical platform. This is not a flow-through chamber, but a chamber where the volumes of reagents (and/or standards) and temperature may be controlled by the program of the sampling/analytical platform. The design and volume of the sample chamber may be optimized to house a particular analytical sensor, or multiple sensors. The ability to control the volumes of solutions introduced into the sample chamber allows for the creation of headspace above the solutions. The creation of a headspace allows analytical sensors to be exposed to atmospheres above the solution for the detection of volatile organic and inorganic compounds, therefore, the analytical platform may accommodate multiple sensors, multiple interrogation components, and multiple sample chambers, depending upon the analytical sensor deployed.

In addition to the “plug and play” analytical sensors and calibration component, the analytical platform is designed to accept a wide variety of sampling methods including, liquid sampling, pumps (peristaltic, diaphragm and centrifugal) and air sampling pumps (FIG. 3). This design allows the analytical platform to sample multiple environmental medias (atmospheres, natural waters and treatment plant effluents) and deliver the samples to multiple sample chambers allowing the same analytical platform to perform very different types of analyses using the system. Sensors are provided between the pump modules and the sample chamber to allow for monitoring water quality parameters for the purpose of low-flow sampling. The water quality sensors at this location are not calibrated but used exclusively to determine when the values of the sensors stabilize. The stabilization of the sensors is an indication that the groundwater sampled is representative of the aquifer and does not represent static water in the wells.

A unique feature of this system is its ability to inject chemicals into the environment. This feature allows the system to inject tracers into the groundwater to measure aquifer parameters, or to measure reagents for site remediation (FIG. 4). The system is capable of collecting, samples from multiple sampling points, such as wells, therefore, the system can inject a solution into one well and collect samples from adjacent wells. An example of an application of a tracer test would be to inject a bromide tracer into the aquifer and collect water samples from adjacent wells, then analyze the water samples with an ion-specific bromide electrode interfaced to the sampling/analytical platform.

The “plug and play” sampling/analytical system may be housed in a variety of structures (trailers, sheds) for environmental protection. The sampling analytical system has the ability to operate with a variety of power sources including line power (120 volts), solar cells, and wind turbine.

The communication between the remote user and the monitoring, system may be accomplished with radio telemetry, cellular or satellite communications (FIG. 1).

### SUMMARY AND ADVANTAGES

The invention disclosed does not use flow-through cells, but interchangeable sample chambers where volumes of reagents, standards, and samples may be precisely introduced into the sample chambers to perform the required analysis. Water level sensors are employed in each sample chamber to deliver a precise volume of sample, reagent or standard. The analyses or calibrations are performed in isolation after all the solutions are injected into the sample chamber. The sample chamber may be stirred, and temperature adjusted by heating or cooling, after being introduced in the sample chamber.

This separation of the sensors, sample chambers, and calibration modules from the sampling/analytical platform is not described in the prior art or literature. The prior art and literature describe elaborate systems containing all the sample chambers, sensors, sampling components, and methods of calibration formed into a single unit. The prior art systems do not describe the ability to quickly exchange a sensor with the accompanying sample chamber and calibration modules within the framework of the sampling/analytical platform. If an alternative sensor is to be deployed with the systems described in the prior art, and the alternative sensor technology is incompatible with the fabricated sample chamber, there does not appear to be an adequate method of quickly adapting the platform to the requirements of the new sensor.

The disclosed monitoring system has the flexibility to assemble multiple chambers, analytical sensors, calibration modules, and sampling methods to match the requirements of a monitoring program. The system does not have any preferred, embodiment except for the method of interconnecting the various boards into a sampling and analytical system. The rigid design documented in the prior art is quite separate from the flexible design presented in this disclosure.

### BRIEF DESCRIPTION OF DRAWINGS

FIG. 1 illustrates the overall monitoring system for remote deployments used to sample groundwater wells.

FIG. 2 illustrates the design of the monitoring system using two chambers, three analytical sensors and three calibration modules connected to four electronic boards.

FIG. 3 illustrates the monitoring system with more than one control board.

FIG. 4 illustrates a monitoring system for the automated performance of a tracer test.

FIG. 5 illustrates the monitoring system configured for the automated performance slug and aquifer tests.

### DETAILED DESCRIPTION

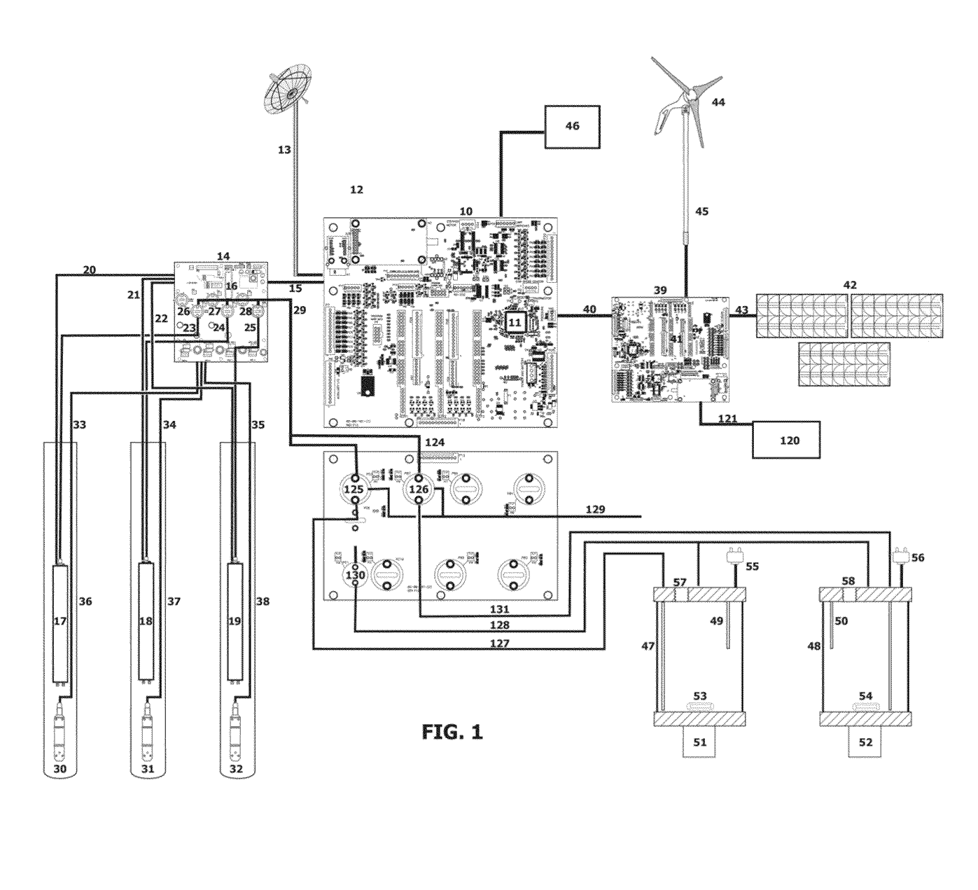

FIG. 1

FIG. 1 illustrates the overall monitoring system. A main control board 10 is the central component of the entire monitoring system. A control microprocessor 11 and communications module 12 are incorporated on the main control board 10. The communication module 12 is connected to an antenna 13. Transmission of signals may be performed using radio telemetry, cellular or satellite methods. A pump control board 14 is connected to the board 10 using a control cable 15. The hoard 14 incorporates a microprocessor 16 to control the operation of various sampling, methods. Multiple selection valves 26, 27, 28 are incorporated on the board 14.

The sampling methods supported by the board 14 include peristaltic pumps and submersible pumps that are directly inserted into monitoring wells 36, 37, 38. The board 14 is connected to multiple pumps 17, 18, 19 located within the multiple monitoring wells 36, 37, 38 by multiple cables 20, 21, 22. The pumps 17, 18, 19 located within the multiple monitoring wells 36, 37, 38 may include electrical turbine and gas-operated diaphragm pumps. The multiple cables 20, 21, 22 are used to conduct electrical signals, electrical power or compressed air depending on the sampling method. Multiple water tubes 23, 24, 25 transport water samples from each of the multiple pumps 17, 18, 19 to the inlets of the multiple selection valves 26, 27, 28 located on the board 14. The outlets of the multiple selection valves 26, 27, 28 are connected into a single sample delivery tube 29. The terminal end of the tube 29 is connected to the inlets of chamber selection valves 125, 126 located on auxiliary board 124. The valves 125, 126 are three-way valves. The common port of the valve 125 is connected to sample chamber 47 with chamber tube 127. The common port of the valve 126 is connected to sample chamber 48 with chamber tube 128. The terminal ends of the tubes 127, 128 are extended to the bottom of the sample chambers 47, 48. The normally-open ports of valves 125, 126 are connected to the waste tube 129.

The multiple sample chambers 47, 48 have multiple volume probes 49, 50, 150 for determining the volume of the sample introduced into the multiple sample chambers 47, 48. FIG. 1 illustrates the two sample chambers, however more than two chambers are possible. The probes 49, 50, 150 may be either optical or conductivity probes. FIG. 1 illustrates one probe in the chamber 48, and two sensors in the chamber 47, additional probes are possible. The chambers 47, 48 have two primary purposes: 1) housing analytical sensors for the analysis of environmental contaminants, and 2) storage of samples for future analysis. Stirring motors 51, 52 are mounted beneath the multiple chambers 47, 48. Magnetic stiffing bars 53, 54 are placed within the multiple chambers 47, 48. Multiple chamber cables with connectors 55, 56 connect the multiple probes 49, 50 and the multiple stirring motors 51, 52 with the main control board 10. The interiors of the multiple chambers 47, 48 are accessed with multiple sensor ports 57, 58. FIG. 1 illustrates one sensor port in each sample chamber, however multiple ports can be required in the design of the sample chamber.

Multiple water level sensors 30, 31, 32 are located in the multiple wells 36, 37, 38. The multiple sensors 30, 31, 32 are connected by multiple electrical cables 33, 34, 35 to the board 14. The multiple sensors 30, 31, 32 are used to measure the water levels in the monitoring wells 36, 37, 38.

An optional power control board 39 is connected with a power control cable 40 to the board 10. The board 39 incorporates a microprocessor 41. The primary function of the power control board is to provide power to the monitoring system when line power (110-volts) is not available. The board 39 is capable of measuring and regulating power from solar panels 42, and/or wind turbine 44 to a battery 120. The board 39 is connected to the solar panels 42 with an electrical cable 43. The board 39 is connected to the wind turbine 44 with an electrical cable 45. The battery 120 is connected to the board 39 with a battery cable 121.

An optional weather station 46 is connected to the board 10 to determine the climatic conditions for the purposes of when to collect water samples.

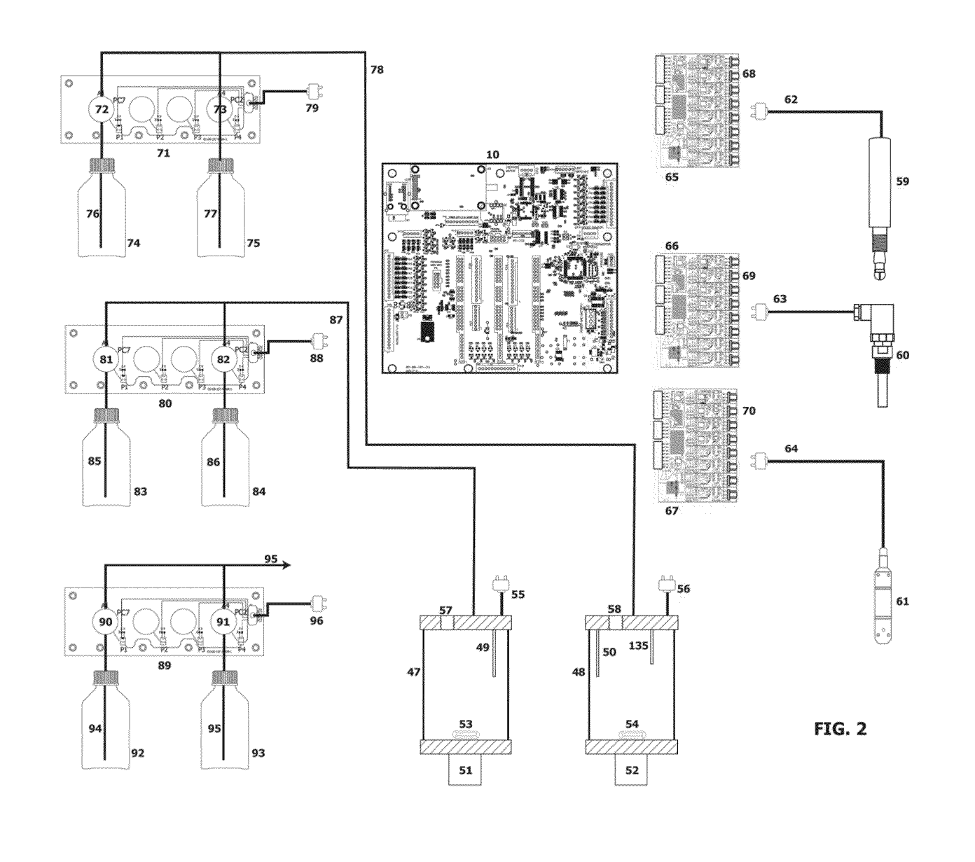

Referring to FIG. 2 illustrates the relationship of the main components of the multiple chamber sampling/analytical system. Multiple analytical sensors 59, 60, 61 are connected by their respective electrical cables and connectors 62, 63, 64 to its respective analytical boards 65, 66, 67. The boards 65, 66, 67 are connected to the board 10. The analytical boards 65. 66, 67 incorporate microprocessors 68, 69, 70. The primary purpose of the individual boards 65, 66, 67 is to convert the raw signals from the analytical sensors 59, 60, 61 into signals proportional to concentrations that can be transmitted to the board 10. The analytical boards 65, 66, 67 are designed for use with specific analytical sensors.

The multiple analytical sensors 59, 60, 61 are connected to the multiple chambers 47, 48 using, multiple sensor ports 57, 58. FIG. 2 illustrates one sensor port in each sample chamber, however, multiple ports can be required in the design of a chamber.

The calibration of the analytical sensors 59, 60, 61 located within the multiple chambers 47, 48 is performed using multiple calibration boards 71, 80, 89. It is typical that one calibration board is dedicated for each analytical sensor incorporated in the monitoring system. Multiple standard selection valves 72, 73 are connected to the calibration board 71. Multiple standard bottles 74, 75 contain low and high standards in FIG. 2. The multiple calibration boards 71, 80, 89 are capable of delivering one to multiple standards. Standard outlet tubes 76, 77 conduct the solutions to the inlet port of the valves 72, 73. Outlets of the valves 72, 73 are connected to a standard delivery tube 78. The tube 78 is connected to the interior of a sample chamber 48. It is typical to use air or inert gas pressure introduced into the headspace of the standard bottles 76, 77 to cause the flow of the standard from the bottles 74, 75 through the valves 72, 73 and into the chamber 47.

The boards 71, 80, 89 are connected to the board 10 using electrical cables with connectors 79, 88, 96.

The number of the boards 71, 80, 89 used in any monitoring system is dependent on the number of the sensors 59, 60, 61 employed in the system.

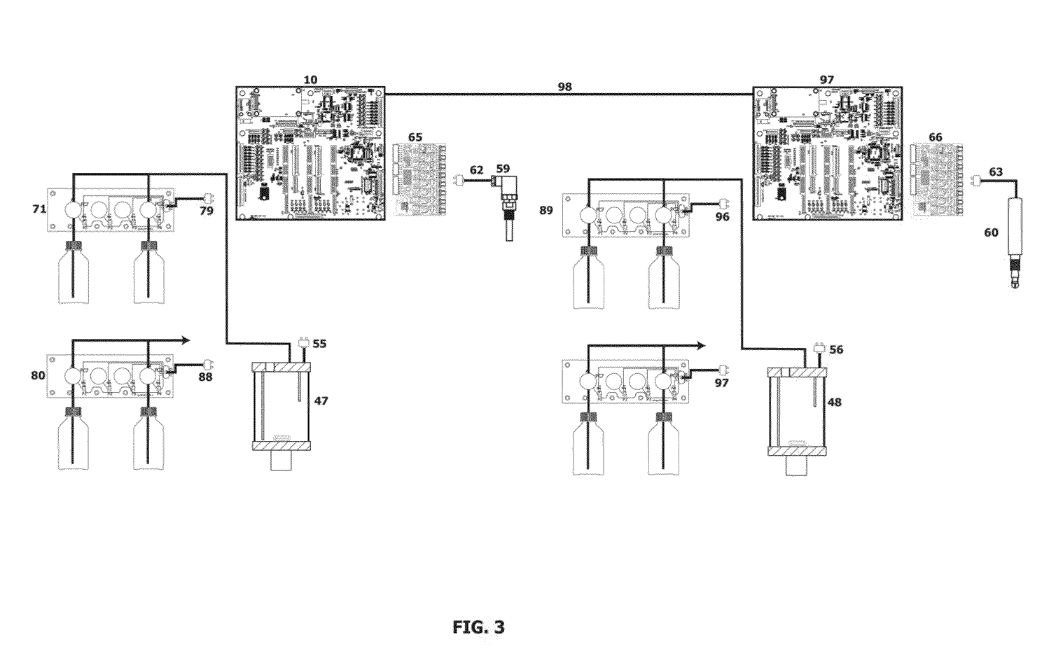

Referring to the FIG. 3

The use of multiple main control boards is presented on the FIG. 3. The board 10 is connected to a second control board 97 with a communication cable 98. The connection of the control boards 10, 97 allow for the expansion of the monitoring system. The board 97 allows the connection of the analytical board 66. The sensor 60 is connected with the cable 63 to the analytical board 66. The board 89 is connected to the board 97 with cable 96. The sample chamber 48 is connected to board 97 with the cable 56. FIG. 3 illustrates the expansion of the monitoring system for accommodating multiple sensors and associated chambers.

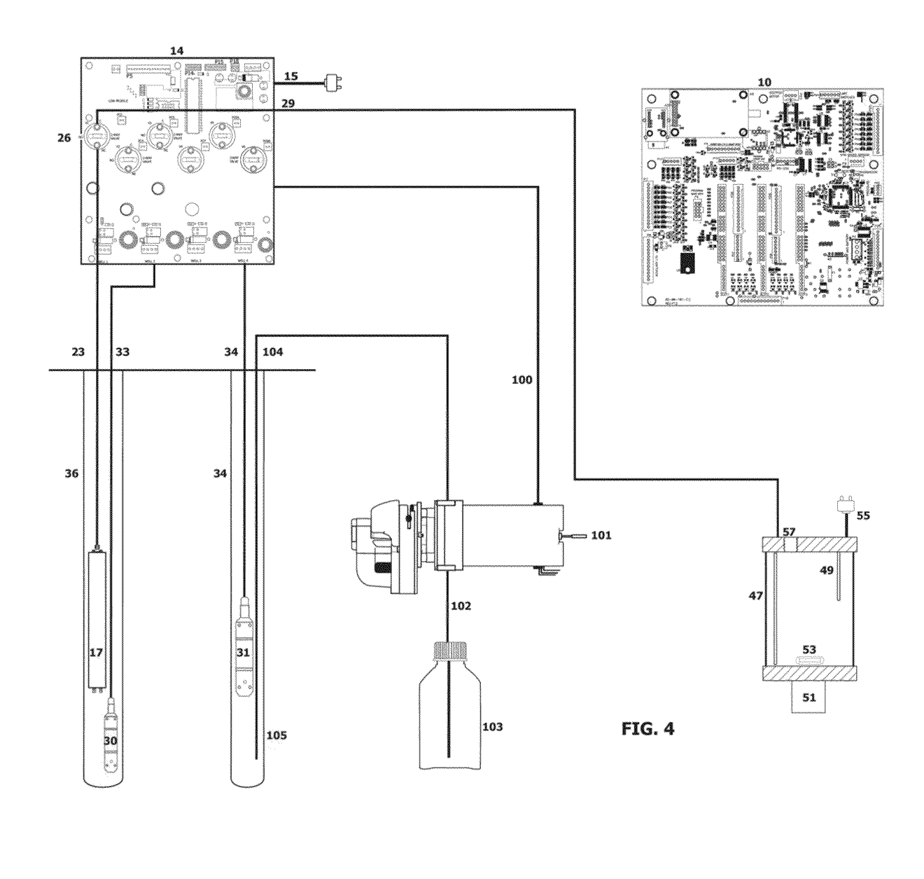

Referring to FIG. 4

The design allows for the control and operation of the chemical monitoring system to be coordinated with the sampling system to allow for tracer tests. A tracer injection pump 101 is connected to a tracer bottle 103 with a tracer inlet tube 102. The outlet of the pump 101 is connected to a tracer outlet tube 104. The tube 104 injects the tracer through its terminal end 105 into the interior of the monitoring well 34. The pump 17 is located in the adjacent monitoring well 36. The tube 23 connects the pump 17 with the valve 26. The tube 29 connects the outlet of the valve 26 with the interior of the chamber 47. The board 14 connects with the board 10 with cable 15. The pump 101 is electrically connected to the board 14 with a cable 100.

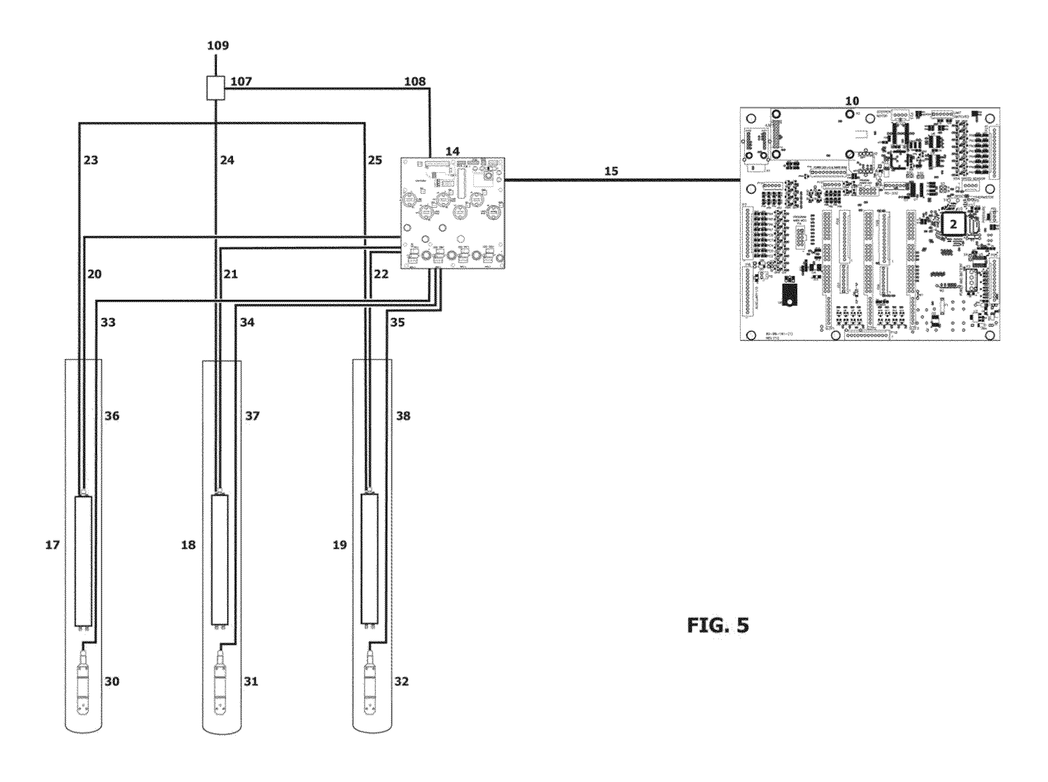

Referring to FIG. 5.

The monitoring system is reconfigured for performing aquifer tests. The control board 10 connects to the pump control board 14 with the cable 15. The board 14 connects to the multiple pumps 17, 18, 19 with the multiple cables 20, 21, 22. The pumps 17, 18, 19 are located within the interiors of the multiple wells 36, 37, 38. The multiple pumps 17, 18, 19 connect to the multiple tubes 23, 24, 25 to a flow meter 107. The flow meter connects to the board 14 with an electrical cable 108. The multiple sensors 30, 31, 32 connects with the multiple cables 33, 34, 35 to the board 14.

FIG. 1 Operation

Referring to the drawing FIG. 1 illustrates the overall monitoring system. A main control board 10 is the central component of the entire monitoring system. The primary operating program is located in the microprocessor 11. The program is used to control the pump control board 14, and the power control board 39. The main control board 10 has the ability to communicate with remote users using multiple communication protocols using the communication module 12 include radio telemetry, cellular or satellite.

The monitoring system collects a sample by the main control board 10 sending a command to the pump control board 14 to select a monitoring well. The pump control board 14 activates the selected pump. The microprocessor 16 controls the pump control board pump 14 and is capable of operating several types of pumps including peristaltic, turbine and diaphragm pumps. If the sampling program selects pump 17 in well 36 then the program of the microprocessor 16 on the board 14 sends the appropriate electrical power, signals or air pressure to operate the selected pump. The activated pump 17 conducts a water sample through an activated valve 26 through the water tube 29. The tube 29 connects to the multiple chamber selection valves 125, 126 located on the auxiliary board 124. The program on board 10 activates the appropriate valve 125, 126 and the water sample transferred into the sample chamber 47, 48. The sample flows into the selected sample chamber 47, 48 until the corresponding water level sensor 49, 50 located within the chambers is satisfied. The program terminates the operation of the pump 17, the valve 26, and valves 125, 126. This action terminates the basic sampling program.

The pump control board 14 collects water level data from the multiple water levels sensors 30, 31, 32 located in each of the monitoring wells 36, 37, 38 during the sampling episode.

The multiple water level sensors 30, 31, 32 are used to measure the water levels in the monitoring wells 36, 37, 38 to determine groundwater flow direction and changes of water level over time. The combination of multiple water level sensors 30, 31, 32 measuring water levels in monitoring wells 36, 37, 38 with the ability to evacuate the wells with the pumps 17, 18, 19 allows for automatically performing low-flow purging of the wells, slug tests and aquifer tests. Automatic low-flow purging is performed by automatically sampling a well without a significant change in the static water level in a monitoring well. The monitoring system automatically collects water level data during the sampling episode. If the sampling rate of the pump causes a decrease in the elevation of the monitoring well, the program decreases the pumping rate until the sampling rate does not disturb the static water level.

An optional power control board 39 and the program contained in the microprocessor 41 monitors the currents and voltages of the solar cells 42, wind turbine 44 and battery 120. The board 39 is used to determine which power source can be used to charge the battery and disconnect the battery to prevent damage from overcharging the battery 120.

An optional weather station 46 is connected to the main control board 10 to determine the climatic conditions for the purposes of when to collect water samples.

FIG. 2. Operation

Referring to drawing FIG. 2 illustrates the relationship of the main components of the multiple chamber sampling/analytical system/calibration system.

The standardization of the analytical sensors located, in the sample chambers can be performed using several types of techniques including:
Calibration curve
Calibration factor

Calibration curve uses the calibration boards 71, 80, 89 to introduce multiple standards in the sample chambers. An example would be the calibration of an analytical sensor located in sample chamber 47 with calibration board 71. The first standard calibration solution is added by the activation of the selection valve 72 to conduct the first standard through the tube 78 into the sample chamber 47. The standard is added until the water sensor 49 is satisfied. The standard is analyzed and then evacuated from the chamber 47. The second calibration solution is added by the activation of the selection valve 73 to conduct the second standard through the tube 78 into the sample chamber 47. The standard is added until the water sensor 49 is satisfied. The standard is analyzed and then evacuated from the chamber 47.

Standardization of the sensor may be performed by the analysis and calculation of a calibration factor. The calibration factor may be calculated from the analysis of sample and spiked sample. The program introduces a sample into the sample chamber and analyzes the sample then evacuates the sample then introduces a sample and adds a known volume and concentration of a standard to the sample. This requires that the volume of the sample and the standard are known to great precision. An example of this type of standardization would be the introduction of a sample from well 36 to sample chamber 47 (FIG. 1). The program activates the pump 17 and the valve 26. The water sample is conducted through tube 23, through valve 26 and tube 29. The valve 125 on board 124 is activated and the sample is introduced into the sample chamber 47. The sample fills the chamber 47 until water level sensor 49 is satisfied. This action terminates the operation of the pump 17, valves 26 and 125 deactivated. The sample is analyzed and the sample evacuated from the sample chamber. The spiked sample is created by the same method as the sample except after the introduction of the sample is completed, a standard is introduced., or spiked, in the sample chamber 47. Referring to FIG. 2 a sample is spiked by the introduction of a known volume of standard into the sample chamber 47. An example is using the calibration board 71 and the standard bottle 74. A standard solution is added by the activation of the selection valve 72 to conduct a standard through the tube 78 into the sample chamber 47. The standard is added until the water sensor 105 is satisfied. The stirring motor 51 agitates a solution in the sample chamber 47. The spiked sample is analyzed and then evacuated from the chamber 47.

It is typical for radiometric analysis detecting trace activities of radioactive isotopes to require several hours to complete the analysis, therefore, it is important if a sample and a spiked sample are to be analyzed, that both samples are collected at the sample time. A second sample chamber is therefore, required to store the sample for later analysis.

FIG. 3. Operation

The operation of the system illustrated on FIG. 3 is similar to FIG. 1 and FIG. 2 except that the chambers 47, 48 and calibration boards 71, 80, 89, analytical boards 65, 66 and analytical sensors 59, 60 are distributed over two control boards 10, 97.

FIG. 4 Operation

The monitoring system on FIG. 4 illustrates an automated tracer test A tracer test consists of the injection of a known concentration and volume of chemical tracer into the aquifer and the collection of samples in adjacent wells to determine if the tracer is present. The monitoring system disclosed is unique in its ability to sample wells analyze the samples, and introduce tracers, reagents, and nutrients in the aquifer.

The board 14 is designed to operate a variety of pumps for the collection of samples from the wells and the injection of tracers and chemicals into the well. The pump 101 is used to inject tracers from the bottle 103 into the monitoring well 34. The tracers flow front the injection well to the adjacent wells. The pump 17 collects water samples in the well 36. A water sample passes through the activated valve 26 through tube 29 and into the sample chamber 47. The sample is analyzed and the concentration of the tracer determined.

FIG. 5 Operation

A slug test is performed b instantaneous removal of a column of water from a monitoring well, and measuring the recharge of the well from the surrounding aquifer. An aquifer test is performed by the removal of water from a central well and the measurement of the response in water levels of the adjacent wells. The monitoring system is configured for an aquifer test in FIG. 5. The well 37 serves as the extraction well. The user sets the program on board 10 and hoard 14 to perform the test. The program activates pump 18 using cable 21. The water from the pump 18 is conducted through the tube 24 to the water meter 107. The rate of water recharge is measured. Water is discharged through the tube 109. Water levels are measured with the sensors 30, 31 in the adjacent wells 36, 37. Signals from the sensors are transmitted to the board 14 with cables 33, 34. The program plots the data of water levels versus time for the calculation of hydraulic conductivity.

We claim:

1. A chemical monitoring system, comprising:
(a) a central control board,
(b) a plurality of chambers,
(c) a plurality of sensors mounted M each of the said chambers,
(d) means of delivering water samples to said chambers,
(e) means of measuring a volume of said water samples delivered to each of the said chambers,
(f) means of calibrating each of the said plurality of sensors mounted in each of the said chambers.

2. Said chemical monitoring system of claim 1 wherein said plurality of sensors may include pH electrode, conductivity electrodes, ORP electrodes, dissolved oxygen electrodes, ion-specific electrodes, colorimetric detector or radiometric detector.

3. Said chemical monitoring system of claim 1 wherein the optical or conductivity detectors are used to measure said volume of water samples delivered to said chambers.

4. Said chemical monitoring system of claim 1 wherein said water samples are delivered to said chambers may include peristaltic, centrifugal or diaphragm pumps.

5. Said chemical monitoring system of claim 1 calibrated with interchangeable calibration modules capable of delivering one to multiple concentrations of standards.

6. Said chemical monitoring system of claim 1 with one or more control boards connected with electrical cables for analysis of contaminants,

7. Said chemical monitoring system of claim 1 combined with sampling system with the ability of determining various aquifer parameters including slug test and aquifer test.

8. Said chemical system of claim 1 comprising a pump and a water level sensor located in the same monitoring well.

9. Said sampling system of claim 1 comprising said pump in said monitoring well and one or more water level sensors located in adjacent wells.

10. Said sampling system of claim 1 comprising, an injection pump for introducing chemicals or nutrients to the aquifer.

11. An automated chemical monitoring system comprising:
(a) an injection pump means of introducing a known concentration of tracer into well,
(b) a sampling pump located in an adjacent well means to collect a water sample and transfer said water sample to a sample chamber,
(c) an analytical sensor located within said sample chamber means for analyzing the concentration of the said tracer in the said water sample.

12. An automated chemical monitoring system comprising:
(a) multiple control boards,
(b) said multiple control boards used to direct water samples into multiple sample chambers,
(c) means of measuring a volume of said water samples delivered to each of the said chambers,
(d) said multiple control boards to acquire data from multiple analytical sensors,
(e) said multiple control boards to calibrate the said multiple analytical sensors
